(19) United States
(12) Patent Application Publication
(10) Pub. No.: US 2004/0236228 A1
(43) Pub. Date: Nov. 25, 2004

(54) Camera containing tool

(76) Inventor: Richard Stoltz, Plano, TX (US)

Correspondence Address: CHALKER FLORES, LLP, 12700 PARK CENTRAL, STE. 455, DALLAS, TX, 75251, US

(21) Appl. No.: 10/850,478

(22) Filed: May 19, 2004

Related U.S. Application Data
(60) Provisional application No. 60/472,071, filed on May 20, 2003.
(60) Provisional application No. 60/471,921, filed on May 20, 2003.

(51) Int. Cl. A61B 6/00

(52) U.S. Cl. 600/473

(57) ABSTRACT

The present invention relates to a system and method of obtaining video images from inside a living body and displaying the images outside the body by interconnecting a video camera extension to a display-containing control unit with a flexible interconnector; inserting the camera extension into the body, wherein the camera extension has a scanned-camera-beam emitter and a reflected-light-collector; illuminating portions of the body with a scanned camera beam from the scanned-camera-beam emitter, causing reflections off the portions of the body; collecting reflection information with the reflected-light-collector and transferring the reflection information through the flexible interconnector to the display system; and displaying the images with the control unit, whereby a relatively inexpensive camera extension with improved depth of field and resolution can be inserted into extremely small openings to generate real-time images of internal body parts.

### CROSS REFERENCE TO RELATED APPLICATIONS

This application claims priority to United States Provisional patent applications entitled, “Camera Containing Medical Tool” Ser. No. 60/472,071 (Docket No. ABI-4); and “In-vivo Tool with Sonic Locator” Ser. No. 60/471,921 (Docket No. ABI-5); both filed May 20, 2003.

### TECHNICAL FIELD OF THE INVENTION

The present invention relates to the in situ, real-time visualization of objects, and more particularly, to an imaging system and method for using fiber optic imaging with enhanced three dimensional imaging during laser ablation.

### BACKGROUND OF THE INVENTION

Endoscopes are currently used to get images from inside the body. Endoscopes typically use a separate flood-light type of illumination and a handle-held, hand-controlled camera or lens to focus the image. An image focused on array of fibers can be optically conveyed outside the body, and when the other end of the fibers outside the body are assembled in the correct order the image can be picked up by, e.g., a video camera. The array of fibers is of course generally much smaller than even the very small video camera. Both of these approaches are fairly expensive and often the devices are contaminated and, if they cannot be economically cleaned, they need to be disposed of. Further, both have lenses with limited focal length.

New experimental surgical techniques are starting to use laser ablation the remove tissue. While most laser machining melts portions of the work-piece, this type of machining is ablative, disassociating the surface atoms. Techniques for generating these ultra-short pulses are described, e.g., in a book entitled “Femtosecond Laser Pulses” (C. Rulliere—editor), published 1998, Springer-Verlag Berlin Heidelberg N.Y. Generally large systems, such as Ti:Sapphire are used for generating ultra-short pulses.

### SUMMARY OF THE INVENTION

The present invention is directed to the use of one or two optical fibers as a camera optical-fiber extension for use with the ablation of a target using a controlled laser ablation system. More particularly, the present invention may use a combination video camera, which can be in color, which detects one or more images through an optical fiber. Control over the camera scan can be changed externally to give the effect of a zoom lens and the camera is not limited by focal length.

The present invention can be a video camera that uses a single optical fiber as a optical-fiber camera-extension. The fiber extension can be inserted into a body (e.g., a human) to obtain images from inside a living body, such that surgery can be performed using an ablation fiber (or tube) inserted along with the in-vivo camera and doing ablation monitored by the camera. The combination may be used to perform operations while viewing the results with insertion of only one or two optical fibers into the body (or e.g., three, if an additional fiber is used to collect reflections and return them to the monitor system).

The ablation fiber, which may be a hollow fiber, generally is slightly larger than the e.g., 125 micron video fiber, but a probe umbilical with the ablation fiber and two (or more) camera fibers can still have an outside diameter that is much less than one-half mm. The camera can also be used on an outer surface of a body. The camera can use a graded-index optical fiber with a camera-beam scanned outside the body and projected into an outside-the-body-end of the graded-index optical fiber, and the inside-the-body-end of the graded-index optical fiber acts as a scanned-camera-beam emitter. The scanned spot of the camera beam illuminates portions of the body, causing spot-by-spot reflections. The camera beam may be generated externally by a relatively low power laser (with, e.g., the ablation beam being generated by a high power laser). Information from reflection is transferring the reflection information through an optical fiber. When the camera-beam scan is synchronized with the display scan, amplified reflection information can form an image displayed, e.g., on a conventional TV. The scan can be changed externally to give the effect of a zoom lens and the camera is not limited by focal length.

The present invention may be used to provide a relatively inexpensive camera with improved depth of field and resolution with an extension fiber that can be inserted into extremely small openings. The fiber extension portion of the camera can be disposable. The ablation laser beam cladding could be used as the reflected-light-collector. Alternatively or additionally, other tools such as a tube for fluid insertion or withdrawal and/or a sonic-locator tube (as described herein) can be inserted with the camera extension. A single optical fiber can be both the camera-beam emitter and its cladding the reflected-light-collector such that only a single optical fiber is required for the camera optical-fiber extension.

Using the present invention, surgery can be performed using an ablation fiber (or tube) inserted along with the in-vivo camera and doing ablation monitored by the camera. In some embodiments, the ablation-beam is scanned inside the body. The ablation-beam can be scanned internally, by moving a mirror with one or two piezoelectric crystals. Alternatively, if a piezoelectric crystal is transparent to the beam, changing a voltage across the crystal can change the angle at which the beam exits the crystal. The camera fiber, however, generally uses an externally scanned beam with the graded-index camera fiber extension preserving the scanning and emitting of the scan beam inside the body. While the scanning is effected by the length of the fiber, this can generally be externally compensated (this type compensation, varying the angle and/or the distance from the fiber axis that a light ray strikes the fiber input end, can also act as a variable telephoto lens).

In some embodiments, the camera-beam scans alternate between one or more of the following visible wavelengths: red, green, and blue or combinations thereof and the reflections are detected by optical detectors of three colors (filtered or unfiltered), but may also be non-visible wavelengths and the display system may be in color of false color; while in other embodiments, an infrared beam is used and variations in the reflections are processed to give an image of an internal part of the body. While lowering the ambient light level may improve the image, absolute darkness is generally not required. A shadow-inducing illumination can also be provided by a secondary light source that is spaced from the scanned-camera-beam emitting optical-fiber-end to provide shadows for improved viewing.

The present invention also includes systems and methods of obtaining video images from inside or the surface of a living body and displaying the images outside the body, the method including the steps of: interconnecting a video camera extension to a display-containing control unit with a flexible interconnector; inserting the camera extension into the body, wherein the camera extension includes a scanned-camera-beam emitter and a reflected-light-collector; illuminating portions of the body with a scanned camera beam from the scanned-camera-beam emitter, causing reflections off the portions of the body; collecting reflection information with the reflected-light-collector and transferring the reflection information through the flexible interconnector to the display system; and displaying the images with the control unit.

The present invention also includes a method of performing a remote procedure, e.g., a surgical procedure, by inserting a tool (e.g., a surgical tool) into or adjacent a remote location (e.g., a living body) and operating the tool from a control unit outside the body, the method including: interconnecting the tool to a control unit containing a display, wherein the interconnection is made through a flexible interconnector; inserting a tool into or adjacent the body containing at least one surgical ablation device, and at least reflected-light-collector and a scanned-camera-beam emitter; illuminating portions of the body by a scanned camera beam from the scanned-camera-beam emitter, causing reflections off the portions of the body; collecting reflection information with the reflected-light-collector and transferring the reflection information through the flexible interconnector to the display system; positioning the tool within the body utilizing the display system; and operating the surgical ablation device from the control unit.

The present invention also includes a method of using a camera to provide images, the method including: projecting a scanned camera-laser beam into an input end of a graded-index optical fiber; projecting a scanned camera-laser-beam from an output end of the a graded-index optical fiber to scan parts of an object to provide light reflections; and collecting at least part of the light reflections in a reflected-light-collector and transferring collected-light information through a flexible interconnector to generate image information. The output end of the graded-index may be inserted inside a body. The image information may be used by a control system.

The present invention also includes a method of using an insertible camera-beam camera to provide real-time images of at least one internal part within a living body, the method including the steps of: interconnecting a camera into the body, wherein the camera comprises a scanned-camera beam emitter and a reflected-light-collector, to a display system, wherein the interconnection is made through a flexible interconnector; inserting the camera into the body; projecting a scanned camera-beam from the camera to scan internal parts of the body to provide successive light reflections; and collecting at least part of the light reflections in the reflected-light-collector and transferring collected-light information through the interconnector to the display system.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The scanned-camera-beam and medical video camera technique of the present invention can use a single optical fiber, e.g., with a 125 micron diameter, inserted into the body to obtain images from inside a living body. When combined with ablation surgical techniques it could perhaps perform operations while viewing the results with insertion of only one or two optical fibers into the body. The ablation fiber, which may be a hollow fiber, generally is slightly larger, the ablation fiber can still have a diameter that is sub-mm. The camera preferably uses a graded-index optical fiber with a camera-beam scanned outside the body and projected into an outside-the-body-end of the graded-index optical fiber, and the inside-the-body-end of the graded-index optical fiber acts as a scanned-camera-beam emitter.

The scanned spot of the camera beam illuminates portions of the body, causing spot-by-spot reflections. Information from reflection is transferring the reflection information through and optical fiber. When the camera-beam scan is synchronized with the display scan, amplified reflection information can form an image displayed, e.g., on a conventional TV. This provides a relatively inexpensive camera with improved depth of field and resolution that can be inserted into extremely small openings. When a pulsed camera beam is the same graded-index optical fiber can be both the scanned-camera-beam emitter and the reflected-light-collector and only a single optical fiber is required.

Surgery can be performed using an ablation fiber (or tube) inserted along with the in-vivo camera and doing ablation monitored by the camera. In preferred embodiments, the flexible interconnector is also used to transfer an ablation beam for in-vivo laser ablation of human tissue. The camera-beam can be scanned moving a mirror, preferably with a piezoelectric crystal, but other mirror positioners, including mirrors mounted on rotating discs, or micro-mechanically movable mirrors, can be used. If a piezoelectric crystal is transparent to the beam, changing a voltage across the crystal can change the angle at which the beam exits the crystal. In some embodiments, the camera-beam scanning crystal is inside the body. Scanning energy to the scanner can be provided by wires or provided through least one optical fiber in the flexible interconnector and electrical energy obtained by a photodetector.

In some embodiments, the camera-beam scans are alternate red, green, and blue wavelengths and the reflections are detected by three optical detectors, and the display system is in color, while in other embodiments, an infrared beam is used and variations in the reflections are processed to give an image of an internal part of the body. A shadow-inducing illumination can also be provided by a secondary light source that is spaced from the scanned-camera-beam emitting optical-fiber-end to provide shadows for improved viewing.

The flexible interconnector connects the camera to a display preferably uses a graded-index optical fiber and the camera-beam is scanned outside the body and projected into an outside-the-body-end of the graded-index optical fiber, and thus the inside-the-body-end of the graded-index optical fiber is the scanned-camera-beam emitter. Thus insertion of one graded-index optical fiber can provide video images from inside a living body that can be displayed outside the body.

The camera-beam is preferably scanned outside the body and projected onto the end of the graded-index optical fiber and transferred into the outside-the-body end of the graded-index optical fiber, through the fiber such that a scanned beam exits an inside-the-body end of the graded-index optical fiber. Where the beam scan is synchronized with the display scan (the scan is of the TV type with a series of vertically offset lines and has the correct timing), the display system can be a conventional TV.

It should be noted that both the scanning beam works especially well with semiconductor-chip laser diodes. Semiconductor-chip diodes can have high efficiency (e.g., about 50%) and have short energy-storage-lifetimes (e.g., a few nanoseconds) and thus can be operated at high repetition rates that give high average output power. Semiconductor-chip diodes can provide a microsecond long train of pulses of nearly constant energy with nanosecond spacing. Thus while other types of lasers could be used, semiconductor-chip diodes are preferred. Note however, that fiber amplifiers, especially when operated at high repetition rates, may also be used.

The ablation works especially well with Cr:YAG lasers, but other types of lasers (e.g., Ti:sapphire) can also be used. These other type of lasers have been used for generating short, high energy pulses, but their efficiencies are low (generally less than 1%) and the pulse energies drop off rapidly when operated at high repetition rates (when they begin to heat up, and when time between pulses becomes short and starts to reduce the time for accumulating energy for the next pulse).

Note that the laser ablation preferably uses a scanned spot. Even with laser ablation, movement such as vibration can cause uneven ablation. Note that other uses such as surgical procedures can use surface ablation or cutting, and can use overlapping ablation to produce a cut surface. In all such uses, a train of pulses is preferably generated by one or more semiconductor-chip diodes. Note also, the train of pulses allows a quasi-CW operation that improves system efficiency, e.g., lessening the number of power up-ramps and down-ramps.

The ablating pulses preferably have a pulse-energy-density of 0.1 to 20 Joules/square centimeter and a pulse-duration of 50 femtoseconds to 1 picoseconds. The pulse-energy-density is more preferably between 0.1 and 8 Joules/square centimeter on the surface being ablated.

A movable camera beam can be used to make an in-vivo TV camera. By using a camera waveguide (e.g., a GRIN fiber) with a beam movable in 2-D with respect to the outside end of the probe generated outside the body, the beam can be made to scan inside the body and reflections from where the beam strikes the body can be picked up by the same and/or one or more other optical fibers and transferred outside the body. The other fibers may have a lens to aid in picking up reflections from the general area to be scanned by the beam, and need not be moved with the beam. An ablation fiber can also be used as a reflection pick-up-fiber.

Graded-index optical fibers can be used to transmit angular information. By cutting the fiber length correctly, a beam entering one end in a plane at an angle from the fiber axis will exit the other end in the same plane at the same angle. Rotating the plane of beam introduction and/or changing the angle will give the same effect on the other end. The fiber will act as a different lens if cut at a different length, however, the length need not be cut precisely, as this can be compensated for by an external variable lens. In any case, at any time, the direction of the spot being illuminated is known, and thus the direction of the reflection is known, and information from the reflections can be processed and displayed on a TV screen.

Conventionally, people have illuminated the entire area to be viewed and either processed reflection information inside the body, or continuously transmitted all reflections from the entire picture outside the body and then processed the picture to display it point-by-point. This improvement illuminates the area one point at a time and transmits the point's reflection (or reflection information) outside the body, and then processes the information to display the picture.

This could be done in color by pulsing the camera with red, green, and blue wavelengths, and it is generally more convenient to have a least one pickup fiber for each color connected to an external sensor for each color (multiple fibers may be connected to a single sensor). It is possible to do this also in at least the near infrared and near ultraviolet as well.

Reflections from a scanned light spot source can be sorted into an image by time (this generally gives the best penetration of a diffusing media). Two-D scanning can be done externally and the angles maintained while transmitting through a fiber.

The scan might or might not be the TV type with a series of lines, but could instead be a series of concentric circles, or a spiral, e.g., starting at the center and spiraling out, and the display could be made by controlling the electron beam of a cathode ray tube to move it in the same pattern, or by using a computer convert the display to be viewed on a conventional TV.

In one preferred embodiment, the camera beam is scanned by applying modulated electric fields across a pair of quartz crystals, with the beam either passing through the crystals or bouncing off a mirror moved by the crystals. Especially with a mirror, other piezoelectrics or even other types of actuators can be used. The same waveguide might (or might not) be used for in-vivo camera ablation of tissue. One or more camera beam-scanning mirrors could be inside the body, but a location outside the body is generally preferred.

Graded-index optical fibers, as noted above, can be used as a lens. Thus the angle of a beam leaving the fiber can be different, but at a known relationship to the beam entering the fiber. Lenses adjusting the beam paths entering the fiber can not only eliminate the need for precisely correct cutting the fiber length, but can also provide zooming of the image.

In another preferred embodiment, an infrared beam is scanned, e.g., to give temperature information. A pulsed camera beam raises the temperature of spot to a detectable level and the reflection is picked up by optical fibers. The frequency and amplitude of the reflection can be measured, and are a function of the spot's temperature before the beam arrived and the known energy in the pulse. As the position of the spot at any given time is known, a thermal image of the area scanned can be generated.

In this approach, the illumination is to be directed. Optical fibers, e.g., graded-index, allow an internal spot to be moved with time (scanned) by external means. Two-D scanning can be done externally and the angles maintained while transmitting through a fiber. Preferably low divergence angle cameras are used to generate the spot. Wavelengths of the spot can be varied. Thus red, green, and blue can be used for a color display. Ultraviolet can be used for a fluorescence display. Infrared can be used for a thermal display.

Reflections can be gathered by one fiber. Multiple, un-oriented, non-critical-length fibers can also be used and their energy easily combined in a single detector. An externally scanned spot makes a much cheaper probe, and endoscope's probes generally need to be disposable.

If a spot of directed illumination is used, received reflections can be sorted into image without angular reflection information. Received reflections are sorted into image by time. Problems of using large lenses and depth of focus are avoided.

The image from this approach can be quite flat. Adding another scanning beam can give a stereo-optic effect and add depth to the image. The use of a different wavelength in the second beam allows reflections from the other fiber-set to be sorted by wavelength or generally eliminated by, e.g., filters. While the image could be viewed stereo-optically (e.g., with glasses with filter-lenses), it may be more convenient for the viewer if the images are combined to produce “shadows” and viewed on a conventional TV-type display. For example, the primary image can be made brighter where picked up from both beams and dimmer where only picked up by only one (e.g., the main) fiber-set. The result would look as if the scene were illuminated a bright light source and some dimmer, diffuse, lighting. It is generally easier to use a general area illumination light source, spaced from the beam source to give the shadow effect. The general area illumination light source can be different wavelengths of red, green and blue the wavelengths of the scanned beam.

The system can be fairly insensitive to ambient light, and the scanning spot is relatively bright and collected light can be filtered to substantially eliminate wavelengths other than the directed-illumination wavelength, but preferably ambient light is minimized.

The camera probe could have a “depth of field” of a meter or more with a resolution of one micron, as the scanning beam can provide an illuminated-spot diameter of about one micron at a point a meter or more away, and the spot reflection may be represented by a single pixel on the display. Generally divergence angle of the scanned beam can be very small (the divergence angle of the camera output can be about 0.1° and the camera spot can be size-reduced and re-collimated with little increase in the divergence angle). Rather than having a true depth of field, this system has spot size that slowly increases linearly with distance, (it can still be only about one mm at 1 kilometer). Further, when the spots begin to overlap, computer analysis can improve the image, or spot characteristics such as size, scan length, or scan rate can be changed.

Presently used endoscopes are not only much larger but also have a short depth of focus to avoid an even bigger lens. Some endoscopes use a lens to focus the image on an array of fibers and then reassemble the fibers in the correct order at the exterior end to get the image, and thus use a large umbilical as well.

When a spot of directed illumination is used, received reflections can be sorted into image without angular reflection information. The line can be stepped in the y-direction direction by time. Alternately, an area of directed illumination can be used, varying both in x-direction and y-direction by wavelength, and received reflections can be sorted into area information without any angular reflection information.

As gathering enough reflected energy can be a problem with present endoscopes, the directed beam can have higher instantaneous illumination levels (even though average levels are lower). Thus the directed beam can work through a longer distance in a diffusing media such as blood. Reflections from a scanned light spot source can be sorted into an image by time (this may have the best penetration of a diffusing media). Two-D scanning can be done externally and the angles maintained while transmitting through a fiber. One-D scanning can be done externally and time-varying wavelengths can provide scanning in the other direction.

Reflections from a light source having a line of light that varies in wavelength with line-position, and in which the line is scanned with time, can be sorted into an image by time and wavelength. If internal scanning is used, this may allow a very small probe.

Reflections from a source of light projecting two planes of light, one plane having one color of light varying in wavelength with vertical-position and a second plane of a second color of light varying in wavelength with horizontal-position can be sorted into an image by the two wavelengths; or adjacent sources of the two color bands, and which could be, e.g., two reds, 600 to 610 horizontally, and 620 to 630 vertically, and similar bands of blue and green for a color display. This can give a continuous picture, rather than a time-scanned picture.

When camera beam is pulsed and the inside-the-body-end of the graded-index optical fiber can be both the scanned-camera-beam emitter and the cladding as a scanned reflected-light-collector. In some embodiments, the reflection is detected by an optical detector within the body that generates an electrical signal, for transfer outside the body.

A TV-like scan can be generated by shining a laser beam onto a wheel with flat mirrors around its periphery such that a series of horizontal lines are reflected and then a larger flat mirror that moves the lines vertically. This raster scan can then be focused by a lens onto the input end of a graded-index fiber, such that the beam exits the output end in a raster scanning mode. In preferred embodiments, the flexible interconnector is also used to transfer an ablation laser-beam for laser ablation of human tissue. The ablation fiber can also be used to gather reflections.

Monitoring of tool location accurately and rapidly is needed to expedite these procedures. A tool that works in combination with these new devices, e.g., using the same element in the umbilical in both locating and operating, can make equipment even smaller. This can be a method of gaining location information of an insertible tool, containing at least one of an ablation probe and a camera probe, within a living body, the method comprising: adding at least one sonic signal-generating device to the insertible tool; inserting the tool into the body; providing energy to the signal-generating device; using the signal-generating device to generate a sonic signal; and using least two external sound detectors to detect the sonic signals to provide information on the location of the insertible tool within the body. The signal or signals can be picked up with conventional medical ultrasonic equipment, and give location of in vivo tools such as an ablator and/or a video camera. The device is sometimes referred to herein as a “pinger”, as it generates a sonic pulse in many applications, however the term “pinger” as used herein is to include either a pulse or a continuous tone. In one embodiment, the method may be used in determining the location of one signal generating device. The use of at least 2 (e.g., 3) sonic devices emitting at different frequencies can be used to give additional information, e.g., orientation when determining the location. Preferably, the signal is an ultrasonic pulse. The body may be a human body. Preferably, at least 3 sonic detectors are used, and the detectors are located on the external surface of the body.

In some embodiments, the signal is an ultrasonic pulse (and preferably generated by a piezoelectric crystal). In some embodiments, the camera is a scanned-laser-beam camera, wherein the laser beam illuminates portions of the body, causing spot-by-spot reflections, and information from reflections is transferring out of the body through an ablation probe optical fiber and the tool contains a camera probe and/or a pinger. The pinger energy may be electrical energy provided by wires extending to the outside of the body or may be pneumatic and provided by a pneumatic tube extending outside of the body and the device may be triggered by hydraulic pressure in a hydraulic tube, which also extends outside of the body. The device can also use a tuning fork or a hydraulic whistle. The energy may be provided by light from an optical fiber, which fiber extends from outside of the body and the light may be detected by an optical detector and energy stored, and a pulse generated when the stored energy built up to a predetermined level, the level may be determined by a Schottky diode, which triggers, e.g., a piezoelectric crystal to generate the pulse.

This can also be a method of gaining information using an insertible tool within a body, the method comprising: inserting a tool containing at least one sonic signal-conveying device into the body; providing energy to the device; using the device to emit a sonic signal within the body; and using least two sound detectors to detect the sonic signals. The sonic signal can be generated externally and the sound conveyed through a hollow tube or a solid metal core surrounded by cladding (preferably the cladding or the hollow tube are of plastic, such that the sound waves travel primarily through the metal and are primarily emitted out the far end). The sound conveyor can have dimensions similar to that of the fiber camera extension.

Note that this not only provides a relatively inexpensive camera that can be inserted into extremely small openings, but also provides an extremely good depth of field and resolution. The camera beam retains its diameter at long distances, and there is no loss of focus, as in conventional cameras. Note the camera is also useful during ablation surgery on the body surface, e.g., mole removal or tattoo removal.

Information of such a system and other information on ablation systems are given in co-pending provisional applications listed in the following paragraphs (which are also at least partially co-owned by, or exclusively licensed to, the owners hereof) and are hereby incorporated by reference herein (provisional applications listed by docket number, title and provisional number).

Docket number ABI-1 Laser Machining—provisional application Ser. No. 60/471,922; ABI-6 “Scanned Small Spot Ablation With A High-Rep-Rate” Ser. No. 60/471,972; and ABI-7 “Stretched Optical Pulse Amplification and Compression”, Ser. No. 60/471,971, were filed May 20, 2003.

ABI-8 “Controlling Repetition Rate Of Fiber Amplifier”—Ser. No. 60/494,102; ABI-9 “Controlling Pulse Energy Of A Fiber Amplifier By Controlling Pump Diode Current”—Serial No. 60/494,275; ABI-10 “Pulse Energy Adjustment For Changes In Ablation Spot Size”—Ser. No. 60/494,274; ABI-11 “Ablative Material Removal With A Preset Removal Rate or Volume or Depth”—Ser. No. 60/494,273; ABI-12 “Fiber Amplifier With A Time Between Pulses Of A Fraction Of The Storage Lifetime”; ABI-13 “Man-Portable Optical Ablation System”—Ser. No. 60/494,321; ABI-14 “Controlling Temperature Of A Fiber Amplifier By Controlling Pump Diode Current”—Ser. No. 60/494,322; ABI-15 “Altering The Emission Of An Ablation Beam for Safety or Control”—Ser. No. 60/494,267; ABI-16 “Enabling Or Blocking The Emission Of An Ablation Beam Based On Color Of Target Area”—Ser. No. 60/494,172; ABI-17 “Remotely-Controlled Ablation of Surfaces”—Ser. No. 60/494,276 and ABI-18 “Ablation Of A Custom Shaped Area”—Ser. No. 60/494,180; were filed Aug. 11, 2003. ABI-19 “High-Power-Optical-Amplifier Using A Number Of Spaced, Thin Slabs” Ser. No. 60/497,404 was filed Aug. 22, 2003.

Co-owned ABI-20 “Spiral-Laser On-A-Disc”, Ser. No. 60/502,879; and partially co-owned ABI-21 “Laser Beam Propagation in Air”, Ser. No. 60/502,886 were filed on Sep. 12, 2003. ABI-22 “Active Optical Compressor” Ser. No. 60/503,659 and ABI-23 “Controlling Optically-Pumped Optical Pulse Amplifiers” Ser. No. 60/503,578 were both filed Sep. 17, 2003.

ABI-24 “High Power SuperMode Laser Amplifier” Ser. No. 60/505,968 was filed Sep. 25, 2003, ABI-25 “Semiconductor Manufacturing Using Optical Ablation” Ser. No. 60/508,136 was filed Oct. 2, 2003, ABI-26 “Composite Cutting With Optical Ablation Technique” Ser. No. 60/510,855 was filed Oct. 14, 2003 and ABI-27 “Material Composition Analysis Using Optical Ablation”, Ser. No. 60/512,807 was filed Oct. 20, 2003.

ABI-28 “Quasi-Continuous Current in Optical Pulse Amplifier Systems” Ser. No. 60/529,425 and ABI-29 “Optical Pulse Stretching and Compressing” Ser. No. 60/529,443, were both filed Dec. 12, 2003.

ABI-30 “Start-up Timing for Optical Ablation System” Ser. No. 60/539,026; ABI-31 “High-Frequency Ring Oscillator”, Ser. No. 60/539,024; and ABI-32 “Amplifying of High Energy Laser Pulses”, Ser. No. 60/539,025; were filed Jan. 23, 2004.

ABI-33 “Semiconductor-Type Processing for Solid-State Lasers”, Ser. No. 60/543,086, was filed Feb. 9, 2004; and ABI-34 “Pulse Streaming of Optically-Pumped Amplifiers”, Ser. No. 60/546,065, was filed Feb. 18, 2004. ABI-35 “Pumping of Optically-Pumped Amplifiers,” was filed Feb. 26, 2004.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of obtaining video images from inside a body and displaying the images outside the body, the method comprising:
interconnecting a video camera extension to a display-containing control unit with a flexible interconnector;
inserting the camera extension into the body, wherein the camera extension comprises a scanned-camera-beam emitter and a reflected-light-collector;
illuminating portions of the body with a scanned camera beam from the scanned-camera-beam emitter, causing reflections off the portions of the body;
collecting reflection information with the reflected-light-collector and transferring the reflection information through the flexible interconnector to the display system; and
displaying the images with the control unit, whereby a camera extension with improved depth of field and resolution can be inserted into small openings to generate real-time images of internal body parts.

2. The method of claim 1, wherein the flexible interconnector comprises a graded-index optical fiber and the camera-beam is scanned outside the body and projected into an outside-the-body-end of the graded-index optical fiber, and wherein an inside-the-body-end of the graded-index optical fiber is the scanned-camera-beam emitter.

3. The method of claim 2, wherein the camera beam is pulsed and wherein the inside-the-body-end of the graded-index optical fiber is both the scanned-camera-beam emitter and the reflected-light-collector, whereby insertion of one graded-index optical fiber can provide video images from inside a living body that can be displayed outside the body.

4. A method of performing surgery by inserting a surgical tool into or adjacent a living body and operating the tool from a control unit remote from the body, the method comprising:
interconnecting the tool to a control unit containing a display, wherein the interconnection is made through a flexible interconnector;
inserting a tool into or adjacent the body containing at least one surgical ablation device, and at least reflected-light-collector and a scanned-camera-beam emitter;
illuminating portions of the body by a scanned camera beam from the scanned-camera-beam emitter, causing reflections off the portions of the body;
collecting reflection information with the reflected-light-collector and transferring the reflection information through the flexible interconnector to the display system;
positioning the tool utilizing the display system; and
operating the surgical ablation device from the control unit.

5. A method of using a camera to provide images, the method comprising:
projecting a scanned camera-laser beam into an input end of a graded-index optical fiber;
projecting the scanned camera-laser-beam from an output end of the graded-index optical fiber to scan parts of an object to provide light reflections; and
collecting at least part of the light reflections in a reflected-light-collector and transferring collected-light information through a flexible interconnector to generate image information.

6. The method of claim 5, wherein the camera-beam is scanned by at least one piezoelectric crystal.

7. The method of claim 6, wherein the piezoelectric crystal moves a movable mirror.

8. The method of claim 6, wherein the piezoelectric crystal is transparent to the beam and changing a voltage across the crystal changes the angle at which the beam exits the crystal.

9. The method of claim 5, wherein the camera-beam is scanned by at least one movable mirror.

10. The method of claim 5, wherein output end of the graded-index is inserted inside a body.

11. The method of claim 5, wherein image information is used by a control system.

12. The method of claim 5, wherein the flexible interconnector comprises a graded-index optical fiber and the camera-beam is scanned outside the body and projected into an outside-the-body end of the graded-index optical fiber, wherein a scanned beam exits an inside-the-body end of the graded-index optical fiber.

13. The method of claim 12, wherein the camera-beam is scanned by a micro-mechanically movable mirror.

14. The method of claim 5, wherein the camera-beam has red, green, and blue wavelengths and the reflections are detected by three optical detectors, and a display system displays an image in color.

15. The method of claim 5, wherein the beam is an infrared beam and the reflections are measured to provide an image of an internal part of the body.

16. The method of claim 2, wherein scan is of the TV type with a series of vertically offset lines, and the display system comprises a conventional TV.

17. The method of claim 5, wherein the reflection is detected by an optical detector within the body to generate an electrical signal, which signal is transferred outside the body.

18. The method of claim 5, wherein the flexible interconnector is also used to transfer an ablation laser-beam for in-vivo laser ablation of human tissue.

19. The method of claim 5, wherein a shadow-inducing-illumination is provided by a secondary light source spaced from the scanned-camera-beam emitting optical-fiber-end.

20. The method of claim 5, wherein a display is provided which has a scan and is synchronized with the beam scan, whereby amplified reflections can form an image which can be displayed on a TV screen.
